(19) United States
(12) Patent Application Publication
(10) Pub. No.: US 2024/0252317 A1
(43) Pub. Date: Aug. 1, 2024

(54) SUTURE LENGTH ADJUSTMENT FOR MINIMALLY INVASIVE HEART VALVE REPAIR

(71) Applicant: NeoChord, Inc., Maple Grove, MN (US)

(72) Inventors: Graham Garvin, Redwood City, CA (US); Joel Helgerson, Boulder, CO (US); Tim Crowley, Arvada, CO (US); Tom Broome, Mound, MN (US); Daryl Edmiston, Draper, UT (US)

(21) Appl. No.: 18/635,638

(22) Filed: Apr. 15, 2024

Related U.S. Application Data
(63) Continuation of application No. 17/524,499, filed on Nov. 11, 2021, now Pat. No. 11,957,584.
Application No. 17/524,499 is a continuation of application No. 16/406,764, filed on May 8, 2019, now Pat. No. 11,173,030.
(60) Provisional application No. 62/669,115, filed on May 9, 2018.

(51) Int. Cl. A61F 2/24; A61B 17/04

(52) U.S. Cl. CPC A61F 2/2457; A61B 17/0401; A61B 17/0487; A61F 2220/0008; A61F 2220/0091

(57) ABSTRACT

Disclosed herein are various embodiments of suture adjustment mechanisms for anchors configured to be inserted into a heart wall of a patient to anchor a suture as an artificial chordae under an appropriate tension for proper valve function. Suture adjustment mechanisms can be configured to retain suture ends extending from the leaflet to the anchor with sufficient force to prevent natural movement of the leaflet from adjusting a length of the suture between the anchor and the leaflet. Free ends of the suture can extend from the anchor external to the body as tensioning strands. A surgeon can supply sufficient force on the tensioning strands from external the body to adjust a length and tension of the suture between the anchor and the leaflet.

### RELATED APPLICATION

This application is a continuation of U.S. application Ser. No. 17/524,499 filed Nov. 11, 2021, which is a continuation of U.S. application Ser. No. 16/406,764 filed May 8, 2019 that is now U.S. Pat. No. 11,173,030, which claims the benefit of U.S. Provisional Application No. 62/669,115 filed May 9, 2018, which is hereby fully incorporated herein by reference.

### TECHNICAL FIELD

The present invention relates to minimally invasive delivery of a suture. More particularly, the present invention relates to anchoring of a suture as an artificial chordae tendineae for a flailing or prolapsing leaflet in a beating heart.

### BACKGROUND

The mitral and tricuspid valves inside the human heart include an orifice (annulus), two (for the mitral) or three (for the tricuspid) leaflets and a subvalvular apparatus. The subvalvular apparatus includes multiple chordae tendineae, which connect the mobile valve leaflets to muscular structures (papillary muscles) inside the ventricles. Rupture or elongation of the chordae tendineae results in partial or generalized leaflet prolapse, which causes mitral (or tricuspid) valve regurgitation. A commonly used technique to surgically correct mitral valve regurgitation is the implantation of artificial chordae (usually 4-0 or 5-0 Gore-Tex sutures) between the prolapsing segment of the valve and the papillary muscle.

This technique for implantation of artificial chordae was traditionally done by an open heart operation generally carried out through a median sternotomy and requiring cardiopulmonary bypass with aortic cross-clamp and cardioplegic arrest of the heart. Using such open heart techniques, the large opening provided by a median sternotomy or right thoracotomy enables the surgeon to see the mitral valve directly through the left atriotomy, and to position his or her hands within the thoracic cavity in close proximity to the exterior of the heart for manipulation of surgical instruments, removal of excised tissue, and/or introduction of an artificial chordae through the atriotomy for attachment within the heart. However, these invasive open heart procedures produce a high degree of trauma, a significant risk of complications, an extended hospital stay, and a painful recovery period for the patient. Moreover, while heart valve surgery produces beneficial results for many patients, numerous others who might benefit from such surgery are unable or unwilling to undergo the trauma and risks of such techniques.

Techniques for minimally invasive thoracoscopic repair of heart valves while the heart is still beating have also been developed. U.S. Pat. No. 8,465,500 to Speziali, which is incorporated by reference herein, discloses a thoracoscopic heart valve repair method and apparatus. Instead of requiring open heart surgery on a stopped heart, the thoracoscopic heart valve repair methods and apparatus taught by Speziali utilize fiber optic technology in conjunction with transesophageal echocardiography (TEE) as a visualization technique during a minimally invasive surgical procedure that can be utilized on a beating heart. More recent versions of these techniques are disclosed in U.S. Pat. Nos. 8,758,393 and 9,192,374 to Zentgraf, which disclose an integrated device that can enter the heart chamber, navigate to the leaflet, capture the leaflet, confirm proper capture, and deliver a suture as part of a mitral valve regurgitation (MR) repair. These minimally invasive repairs are generally performed through a small, between the ribs access point followed by a puncture into the ventricle through the apex of the heart. Although far less invasive and risky for the patient than an open heart procedure, these procedures still require significant recovery time and pain.

Some systems have therefore been proposed that utilize a catheter routed through the patient's vasculature to enter the heart and attach a suture to a heart valve leaflet as an artificial chordae. While generally less invasive than the approaches discussed above, transcatheter heart valve repair can provide additional challenges. For example, with all artificial chordae replacement procedures, in addition to inserting a suture through a leaflet, the suture must also be anchored at a second location, such as at a papillary muscle in the heart, with a suture length, tension and positioning of the suture that enables the valve to function naturally. If the suture is too short and/or has too much tension, the valve leaflets may not properly close. Conversely, if the suture is too long and/or does not have enough tension, the valve leaflets may still be subject to prolapse. Proper and secure anchoring of the suture away from the leaflet is therefore a critical aspect of any heart valve repair procedure for inserting an artificial chordae. However, adjusting length of a suture in a transcatheter procedure is difficult as it is not possible for the surgeon to physically control the suture and its length once the suture is in the heart.

### SUMMARY

Disclosed herein are various embodiments of suture adjustment mechanisms for anchors configured to be inserted into a heart wall of a patient to anchor a suture as an artificial chordae under an appropriate tension for proper valve function. Suture adjustment mechanisms can be configured to retain suture ends extending from the leaflet to the anchor with sufficient force to prevent natural movement of the leaflet from adjusting a length of the suture between the anchor and the leaflet. Free ends of the suture can extend from the anchor external to the body as tensioning strands. A surgeon can supply sufficient force on the tensioning strands from external the body to adjust a length and tension of the suture between the anchor and the leaflet.

In one embodiment, a suture adjustment mechanism is embodied in an anchor configured to be implanted into a patient's heart wall to anchor a suture extending from a valve leaflet of the heart as an artificial chordae. The anchor can include an anchor body and a means for retaining a suture within the anchor body. The means for retaining a suture is configured to enable adjustment of a length of the suture extending between the anchor body and the valve leaflet by pulling tensioning strands of the suture extending from the anchor body out of the patient's heart while preventing forces applied on the length of the suture extending between the anchor body and the valve leaflet due to movement of the leaflet from adjusting the length of the suture extending between the anchor body and the valve leaflet.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1K depict various steps in a method of anchoring a suture in a beating heart of a patient to function as an artificial chordae according to an embodiment.

FIGS. 2A-2B depict steps in a method of anchoring a suture in a beating heart of a patient to function as an artificial chordae according to an embodiment.

FIGS. 3A-3F depict a suture length and tension adjustment mechanism according to an embodiment.

FIGS. 4A-4C depict a suture length and tension adjustment mechanism according to an embodiment.

FIG. 5 depicts a suture length and tension adjustment mechanism according to an embodiment.

FIGS. 6A-6B depict a suture length and tension adjustment mechanism according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure is generally directed to adjusting a length and/or tension of sutures inserted as artificial chordae into one or more heart valve leaflets through an intravascular, transcatheter approach. A heart valve leaflet may be captured and a suture inserted through the leaflet in any manner known in the art. One such leaflet capture catheter and procedure is disclosed in copending U.S. Utility patent application Ser. No. 16/363,701, which is hereby incorporated by reference herein. Another transcatheter procedure for inserting an artificial chordae is disclosed in U.S. Patent Publication No. 2016/0143737, which is hereby incorporated by reference herein.

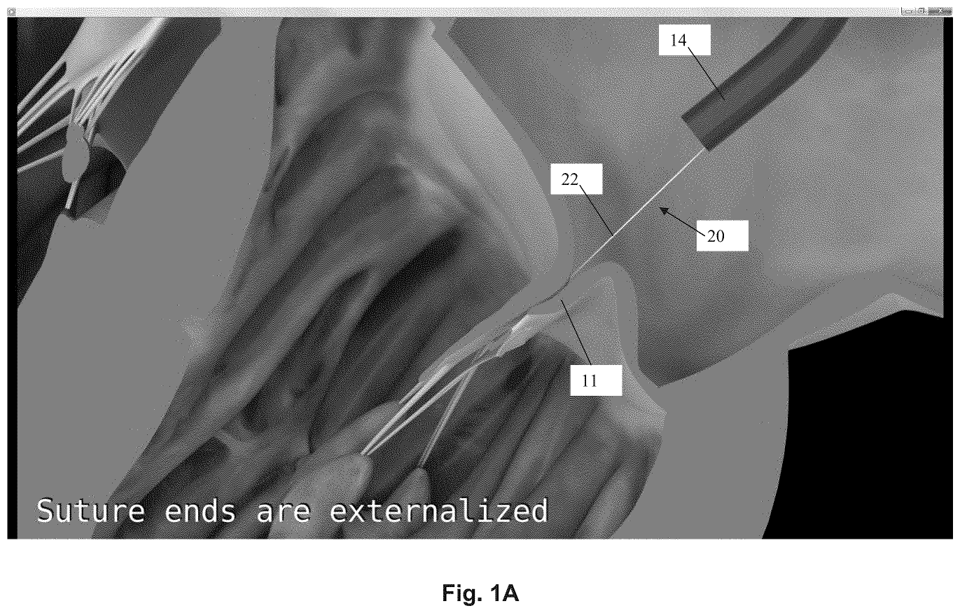

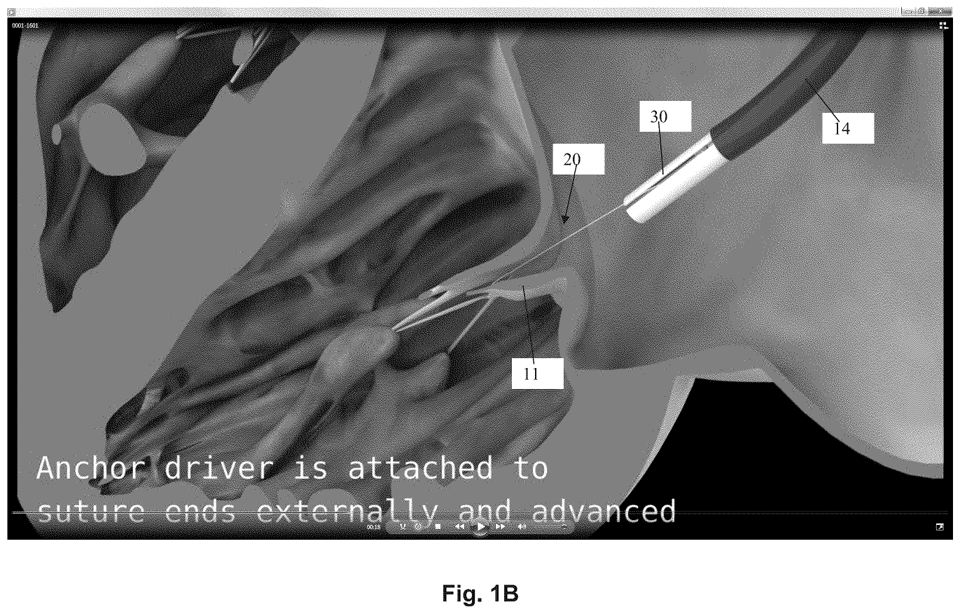

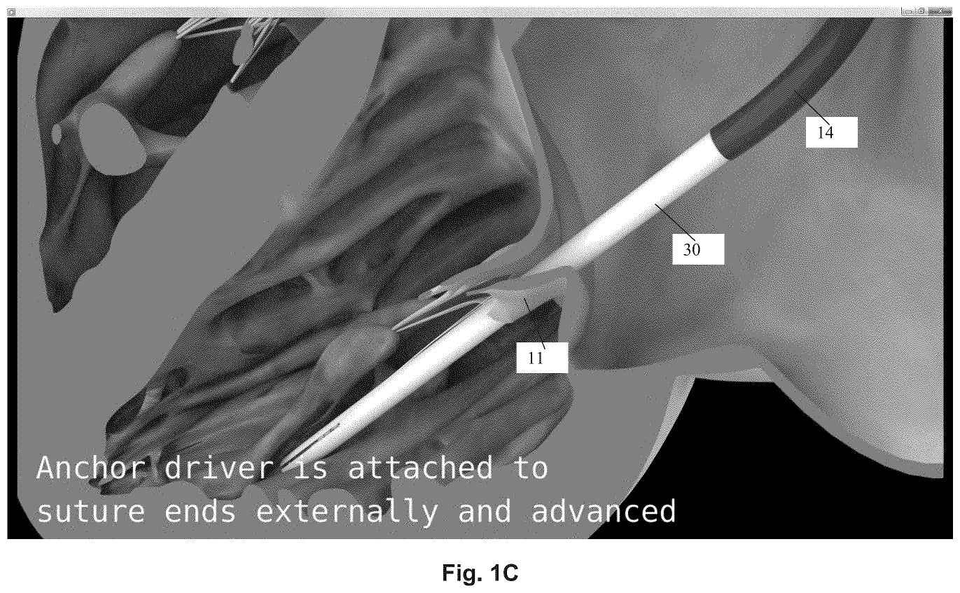

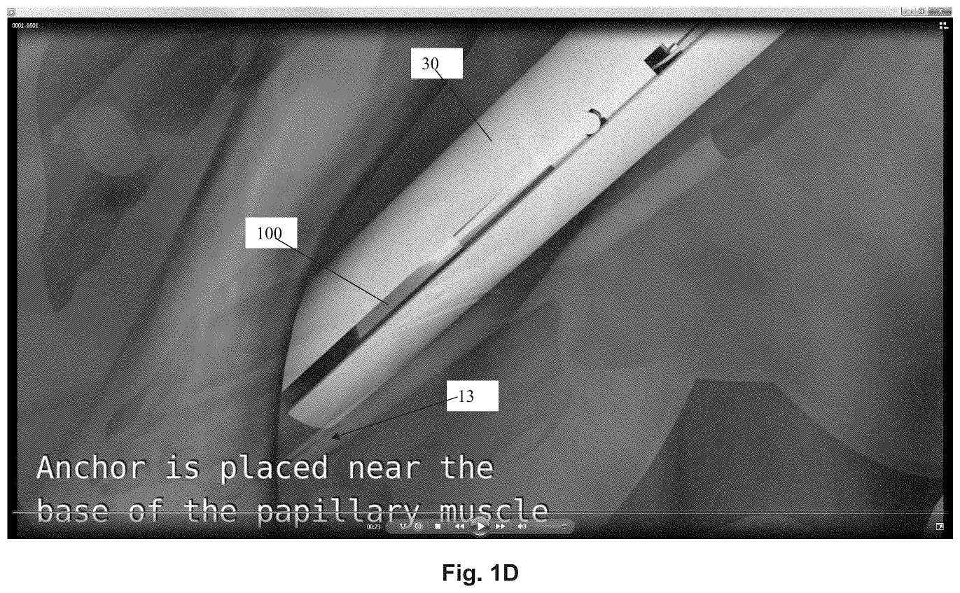

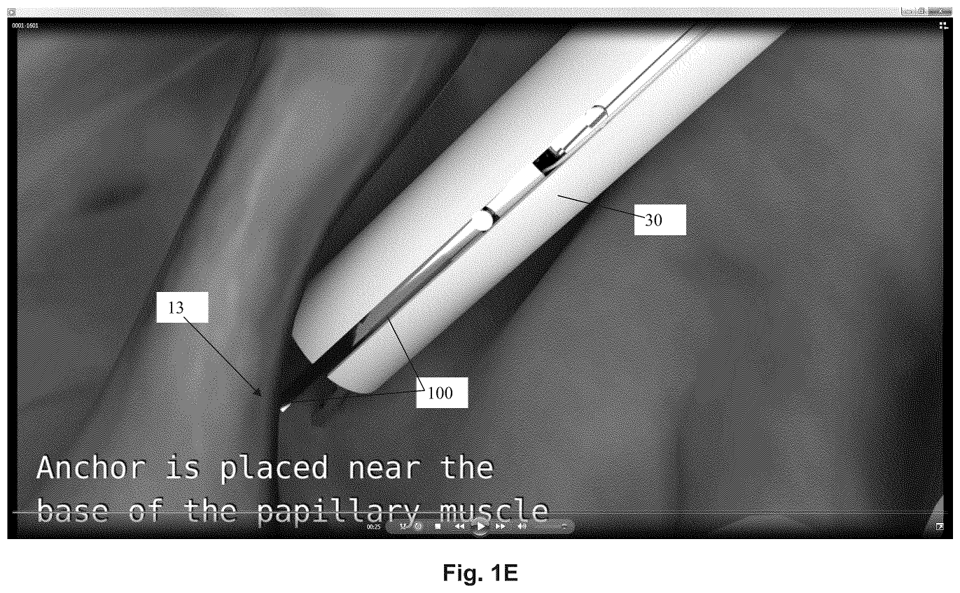

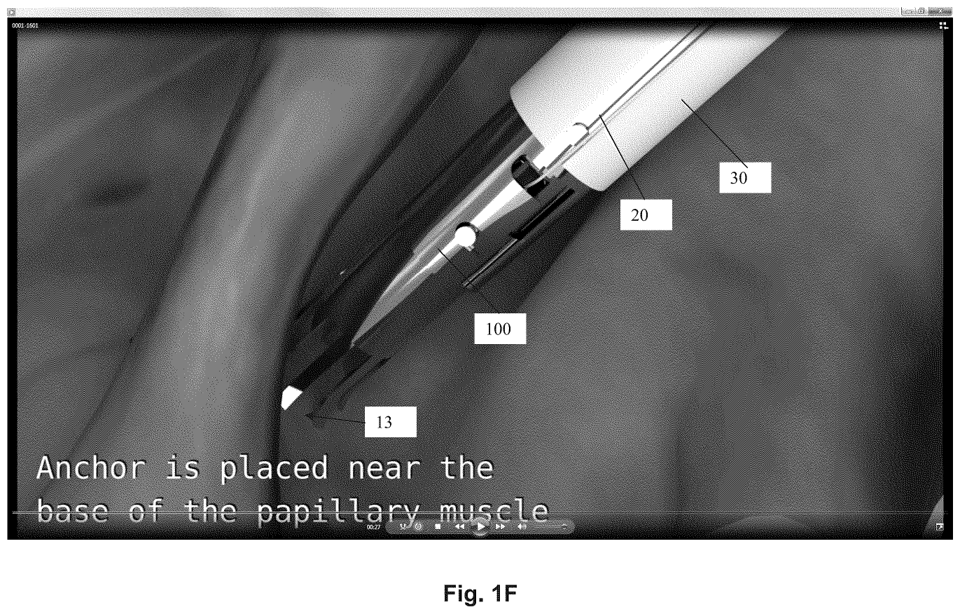

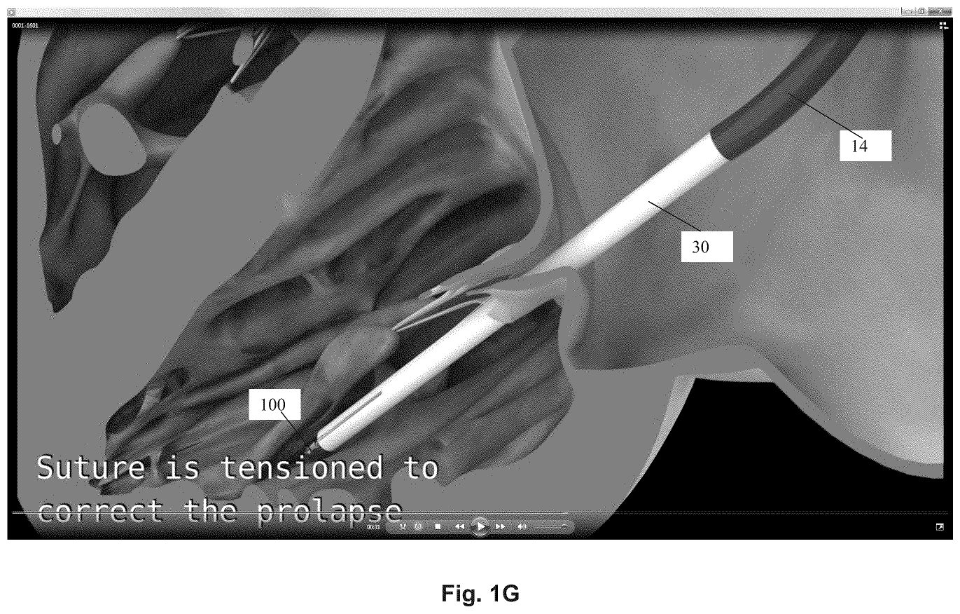

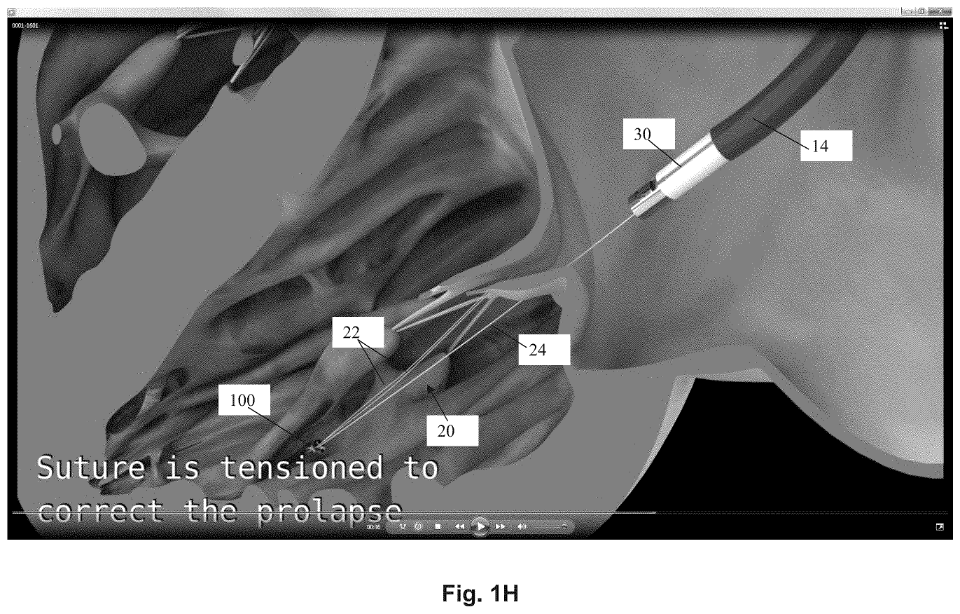

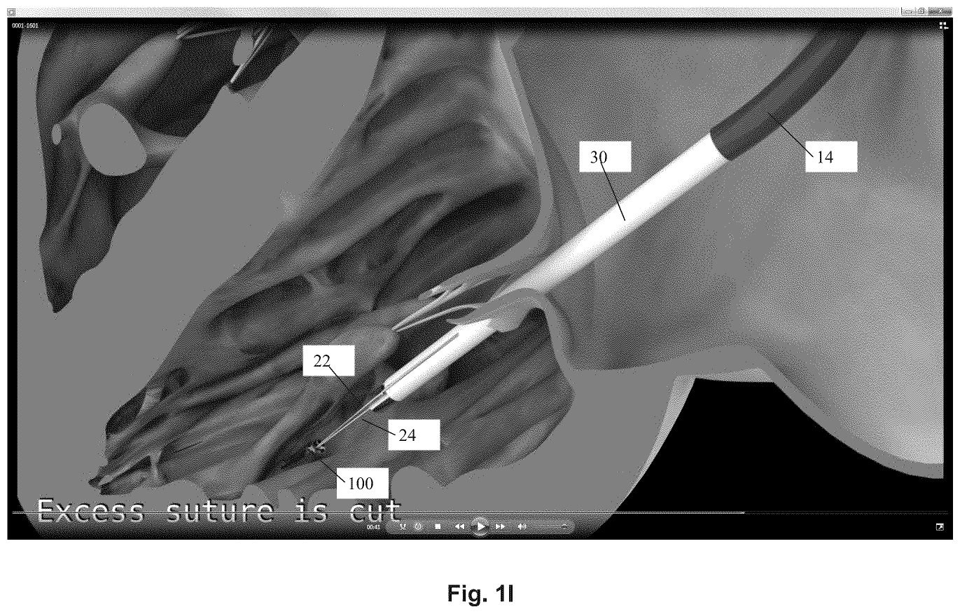

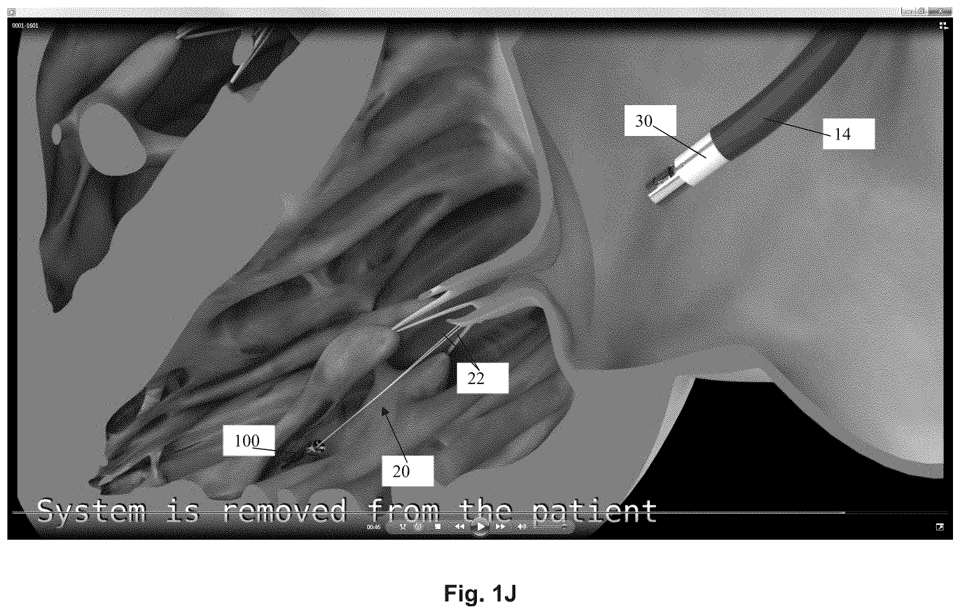

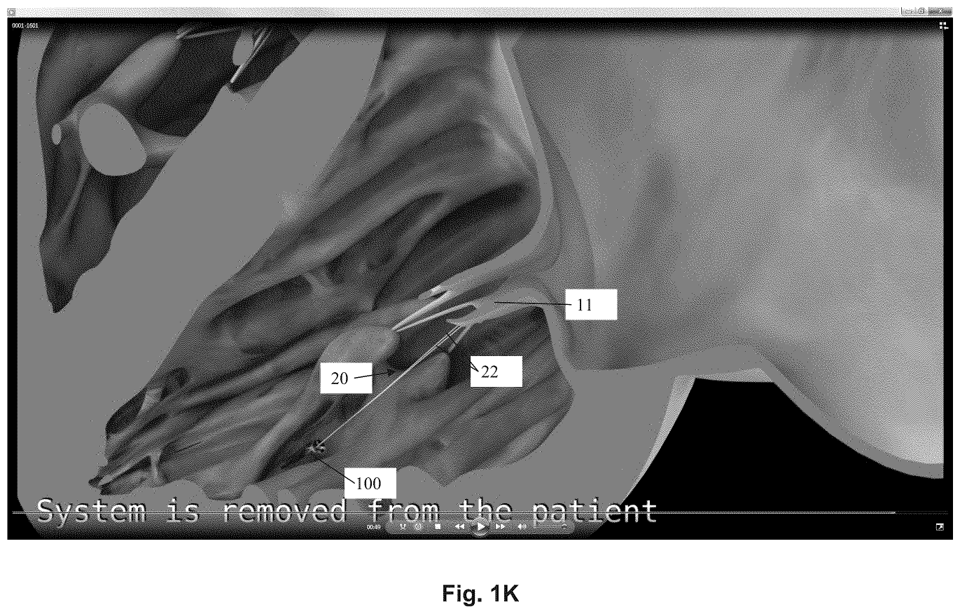

Referring to FIGS. 1A-1K, a procedure for anchoring a suture inserted as an artificial chordae in a transcatheter procedure on a beating heart of a patient following insertion of the suture into a leaflet is schematically depicted. In this embodiment, a loop of suture has been inserted through the leaflet and the two free ends of the suture then inserted through the loop to form a girth hitch knot around the edge of the leaflet. Further detail regarding attaching a suture to a leaflet in this manner can be found in U.S. Patent Publication No. 2017/0290582, which is hereby incorporated by reference herein.

Following insertion of the suture 20 into the leaflet 11, the deployment catheter used to insert the suture is withdrawn through the guide catheter 14 and the two free ends 22 of the suture 20 are also withdrawn external to the body. The suture ends 22 are then attached to an anchor contained in an anchor driving catheter 30. Alternatively, the anchor could be pre-attached to the suture prior to insertion of the suture into the leaflet. The anchor driving catheter 30 is inserted into the guide catheter 14, routed through the catheter into the body and advanced passed the leaflet 11 to the heart wall 13 below the valve at, for example, a papillary muscle as shown in FIGS. 1B-1D. The anchor driving catheter 30 is then used to insert the anchor 100 into the myocardium as shown in FIGS. 1D-1G and as described in more detail below. Various embodiments of such anchors can be found in U.S. Provisional Patent Application Nos. 62/669,096 and 62/669,123, entitled Low Profile Tissue Anchor for Minimally Invasive Heart Valve Repair and Radial Arm Tissue Anchor for Minimally Invasive Heart Valve Repair, respectively, both of which are hereby incorporated by reference herein in their entireties.

After insertion of the anchor 100 into the heart tissue, the anchor driving catheter 30 is withdrawn to a position superior of the valve as shown in FIG. 1H and the length and tension of the suture ends 22 extending from the leaflet 11 are tested and adjusted until it is determined that normal valve function has been achieved. This determination can be made through use of ultrasonic imaging, for example. The tension is adjusted through a tensioning strand 24 of the suture depicted in FIG. 1H. Once the proper length and tension has been determined using, for example, transesophageal echocardiography or other non-invasive methods, the anchor driving catheter 30 is advanced back down along the tensioning strand 24 and to sever the strand at the anchor 100, or a separate device configured to sever the strand can be used. The entire catheter system, including the anchor driving catheter 30 and the guide catheter 14 is then withdrawn from the patient's body. Referring to FIG. 1K, the suture 20 remains in the body extending between the leaflet 11 and the anchor 100 to function as an artificial chordae tendonae.

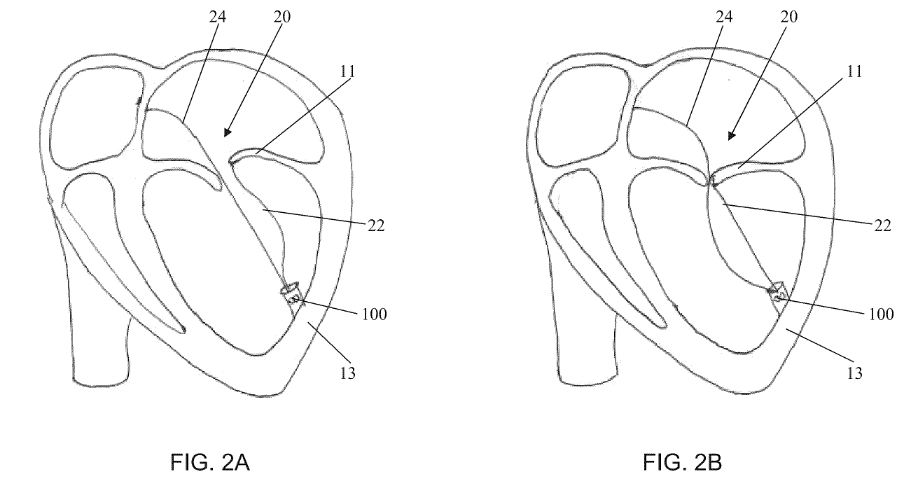

FIGS. 2A-2B depict further details regarding adjustment of the length and tension of the suture 20 according to an embodiment. After initial insertion of the anchor 100 into the heart wall 13, the free ends 22 of the suture 20 extend between the anchor and the leaflet 11 and the tensioning strand 24 of the suture 20 extends out of the body where it is accessible to the surgeon, as described above. FIG. 2A depicts one example of an initial configuration prior to suture adjustment. In the depicted configuration, a length of the suture ends 22 between the anchor 100 and the leaflet 11 is too long such that there is not enough tension on the leaflet 11, which could cause the leaflet 11 to continue to prolapse. FIG. 2B depicts a final configuration after suture adjustment. In this configuration, the length of the suture ends 22 between the anchor 100 and the leaflet 11 have been shortened to provide a tension that enables the leaflet 11 to coapt with the other leaflet during systole while preventing the leaflet 11 from prolapsing. Once proper suture length and tension has been achieved as shown in FIG. 2B, the tensioning strand 24 can be severed as discussed above.

Disclosed herein are various embodiments of mechanisms that can be employed to adjust the length and/or tension of a suture as an artificial chordae in procedures such as those described above. Such mechanisms enable suture adjustment from outside the body in a transcatheter, intravascular procedure.

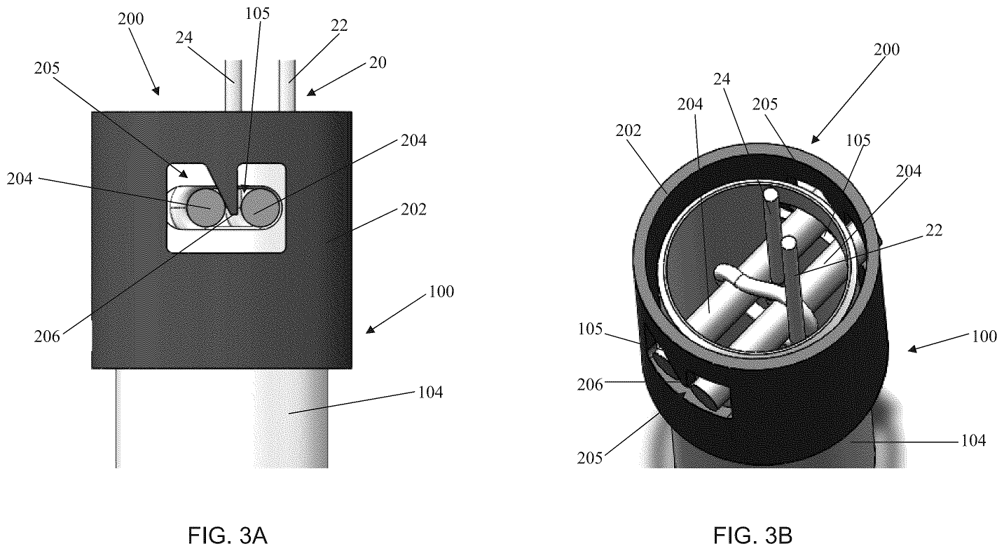

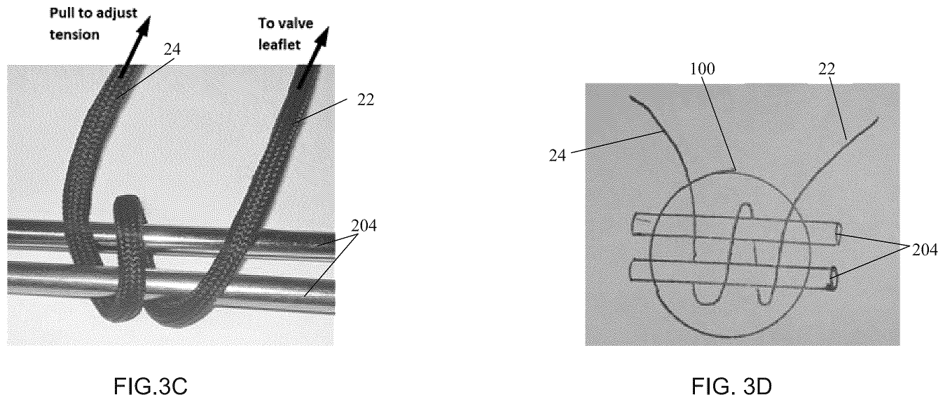

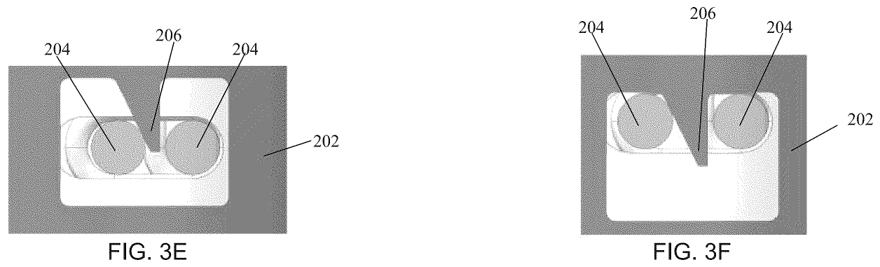

FIGS. 3A-3E depict a suture adjustment mechanism 200 of a suture anchor 100 according to an embodiment. Suture adjustment mechanism 200 can be disposed adjacent a proximal portion of an anchor body 104 of suture anchor 100. For sake of clarity, only body portion 104 of suture anchor 100 is depicted. Although not shown in these figures for sake of clarity, suture anchor 100 would further include a distal portion for embedding the anchor into heart tissue, such as, for example those disclosed in the toggle anchor and spider anchor applications incorporated by references above.

Suture adjustment mechanism 200 includes an actuation tube 202 and a pair of pins or bars 204. Actuation tube 202 can surround a portion of anchor body 104 of anchor 100. Bars 204 can extend transversely across a long axis of anchor body 104. Bars 204 can extend through and be supported within opposing apertures 105 through anchor body 104. Bars 204 can further extend through and be supported within opposing apertures 205 in the actuation tube 202 adjacent the apertures 105 in the anchor body 104. In the depicted embodiment, bars 204 can seat within apertures 105, 205, in a generally horizontal side-by-side and parallel configuration with each other. An actuation projection 206 on one or both sides of the actuation tube can extend from actuation tube 202 into the aperture 205 and can be selectively positioned between pins, as will be discussed below.

A suture 20 can extend into anchor body 104 and be wrapped around pins/bars as shown in more detail in FIGS. 3B-3D. For sake of clarity, it is noted that only a single suture end is shown in these Figures. However, it should be noted that each suture inserted through a leaflet will have a pair of suture ends attached to the suture adjustment mechanism 200. As shown in these figures, each suture end 22 extending from the leaflet to the anchor 100 wraps completely around both pins/bars. The suture 20 then extends between the bars 204 as tensioning strands 24 extending back out of the body to the surgeon, as described above.

The actuation projection 206 of actuation tube 202 can be biased to a proximal position as shown in FIG. 3E and can be actuated downwardly as shown in FIG. 3F to drive the bars 204 apart. In use, when anchor 100 is attached to the anchor delivery catheter 30, the delivery catheter 30 can interface with the actuation tube 202 to actuate the projection 206 downwardly to force the bars 204 apart. This space between the bars 204 enables the anchor 100 to slide along the tensioning strand 24 from a position outside the body where the anchor 100 is attached to the suture 20 to within the heart adjacent the myocardium where the anchor 100 is to be inserted. Following insertion of the anchor 100, the anchor delivery catheter 30 is withdrawn as discussed above. Once the anchor delivery catheter 30 is detached from the actuation tube 202, the tube returns to the proximal position shown in FIG. 3E and the tensioning strand 24 of the suture 20 is held between the bars 204 with sufficient force such that the forces applied on the free ends 22 of the suture (wrapped around the bars 204) by the natural movement of the leaflet to which the suture is attached are not sufficient to move the suture. However, a surgeon operating the tensioning strands 24 of the suture 20 external to the body can supply sufficient force by pulling on the tensioning strands 24 to shorten the distance between the anchor 100 and the leaflet (see FIGS. 2A-2B). The actuation tube can be utilized to release the suture tension applied to the valve leaflet in a situation where increasing the length of the suture between the anchor and the leaflet is desired by the surgeon following the assessment of the valve function. Once the tension and length of the suture 20 have been properly adjusted and the tensioning strand 24 is severed as discussed above, the suture length will be fixed.

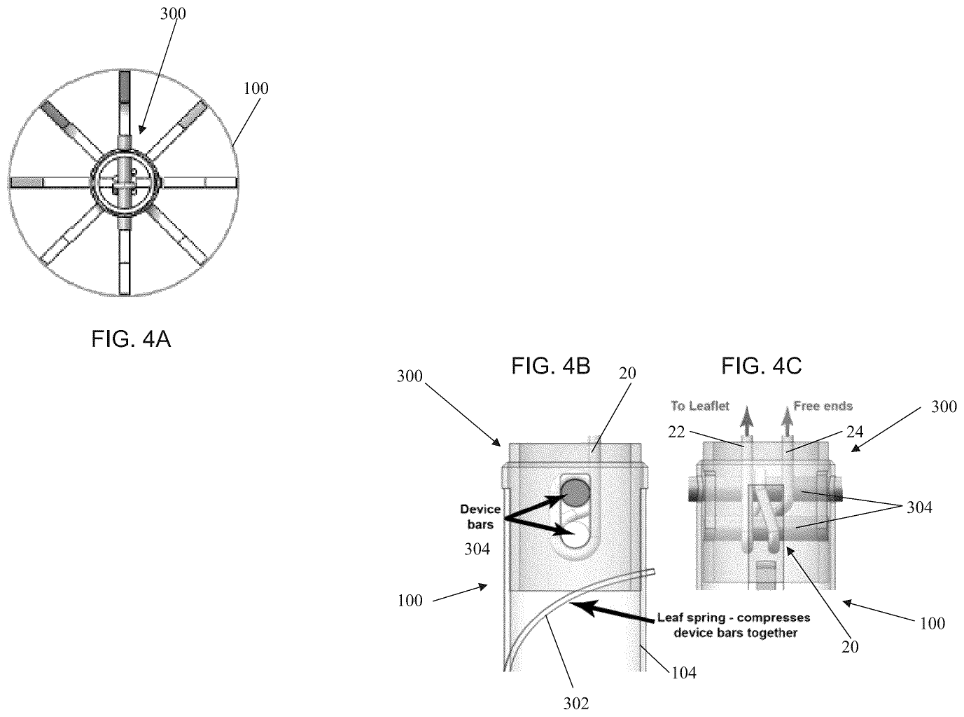

FIGS. 4A-4C depict a suture adjustment mechanism 300 according to another embodiment. Suture adjustment mechanism 300 is similar to suture adjustment mechanism 200 in that the suture 20 wraps around a pair of bars or pins 304 contained in a proximal portion of an anchor body 104 of suture anchor 100. In contrast to the generally horizontally aligned bars 204 of suture adjustment mechanism 200, the bars 304 of suture adjustment mechanism 300 are oriented and aligned generally vertically with respect to each other. Bars 304 can extend through the anchor body 104 similar to the embodiment described above.

The suture ends 22 extending between the leaflet and the anchor 100 wrap around the bars 304 and the free ends or tensioning strands 24 extend between the bars 304, as shown in FIGS. 4B-4C. A leaf spring 302 can be positioned within anchor body 104 to apply a force to bias the bars together, either by directly contacting the lower bar 304 or by contact a housing element that contains the pins. Tension on the suture and the length between the anchor 100 and the leaflet can be adjusted by pulling on the tensioning strands 24 outside the body with sufficient force to pull the suture 20 through the bars 204 and shorten the connection to the leaflet. In one embodiment, the force required by the surgeon to pull the suture through the mechanism is 0.1 pounds. Natural forces of the leaflet on the suture ends 22 are not sufficient to pull the suture through the bars 304, and because the suture ends 22 are wrapped around the pins 304 such forces increase the inward clamping force provided by bars 304 to the tensioning strands 24 positioned therebetween. Tension can be released by exerting force downward onto leaf spring to enable the pins to spread apart. Such a force can be applied by, for example, a catheter such as delivery catheter 30 as the anchor 100 is advanced from external the body along the suture 20 to the myocardium where it is to be anchored.

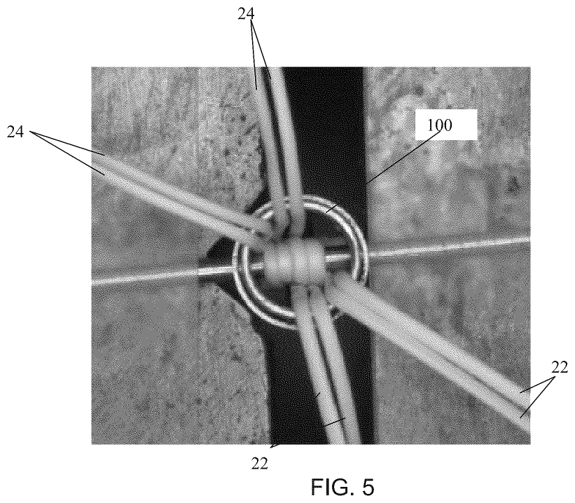

As noted above, in the previous embodiments only a single suture end has been shown for sake of clarity. In practice, each suture will have a pair of ends 22 extending from the leaflet and a corresponding pair of tensioning strands 24. In one embodiment, each anchor 100 and corresponding tensioning mechanism 200, 300 can accommodate a pair of sutures connected to the leaflet, which therefore includes two pairs (4) of suture ends 22 extending to the leaflet and two pairs (4) of tensioning strands 24 extending out of the body, as depicted schematically in FIG. 5.

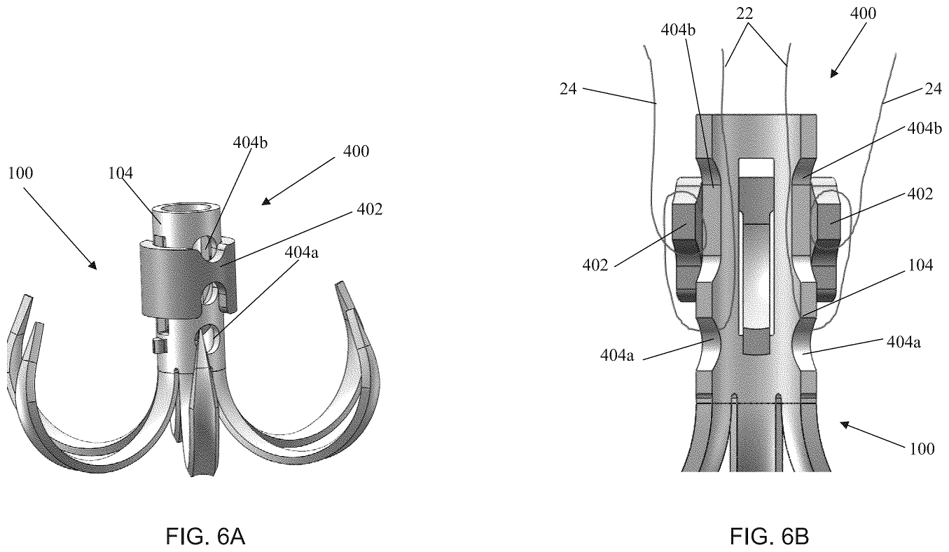

FIGS. 6A-6B depict a suture adjustment mechanism 400 defined in an anchor body 104 of an anchor 100 according to a further embodiment. Essentially, this embodiment replaces the pins or bars 204, 304 of the previous embodiments with apertures 404 formed through opposing sides of the anchor body 104. In one embodiment, apertures 404 include a distal aperture 404a and a proximal aperture 404b. In the depicted embodiment, the distal apertures 404a are generally circular and the proximal apertures 404b are elongate. Suture adjustment mechanism 400 can further include a suture wrapping sleeve 402. Suture wrapping sleeve 402 can be positioned around the elongate proximal apertures 404b.

Referring to FIG. 6B, each suture end 22 of a suture 20 extending from the leaflet enters into anchor body 104 through an open proximal end of anchor body 104 and extends into a hollow interior of anchor body 104. The suture ends 22 then extend out of the distal apertures 404a and are wrapped around the suture wrapping sleeve 402 at the elongate proximal apertures 404b. The suture 20 then extends back out of the body as a pair of tensioning strands 24 for adjusting a length of each respective suture end 22 relative to the leaflet. As with previous embodiments, a surgeon can provide sufficient force on the tensioning strands 24 to adjust the length and tension of the suture ends 22, but the natural forces of the leaflet are insufficient to adjust the wrapped suture ends 22.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

What is claimed is:

1. An anchor configured to be implanted into a heart wall of a heart of a patient to anchor a suture extending from a valve leaflet off the heart as an artificial chordae, comprising:
an anchor body having an open interior;
a distal portion configured to be embedded into heart tissue;
a spring disposed within the open interior of the anchor body, wherein when the spring is compressed a suture disposed in the anchor body can slide freely within an open space in the anchor body to adjust a tension on the suture and when the suture is in a released state the spring exerts a force eliminating the open space to clamp the suture at the tension within the anchor body.

2. The anchor of claim 1, further comprising a movable housing element disposed within the anchor body configured to be moved when the spring is released to clamp the suture.

3. The anchor of claim 1, further comprising a suture clamp disposed within the anchor body, wherein the suture clamp is configured to clamp the suture at the tension when the spring is released.

4. The anchor of claim 1, wherein the suture is clamped adjacent a proximal end of the anchor opposite of the distal portion.

5. The anchor of claim 1, wherein the spring is configured to be selectively compressed and released to enable re-tensioning of the suture.

6. An anchor configured to be implanted into a heart wall of a heart of a patient to anchor a suture extending from a valve leaflet off the heart as an artificial chordae, comprising:
an anchor body having an open interior;
a distal portion configured to be embedded into heart tissue;
a spring disposed within the open interior of the anchor body, wherein the spring can be selectively compressed to enable adjustment of a tension of suture extending into the open interior of anchor body and released to clamp the suture at the tension within the opening interior of the anchor body.

7. The anchor of claim 6, further comprising a movable housing element disposed within the anchor body configured to be moved when the spring is released to clamp the suture.

8. The anchor of claim 6, further comprising a suture clamp disposed within the anchor body, wherein the suture clamp is configured to clamp the suture at the tension when the spring is released.

9. The anchor of claim 6, wherein the suture is clamped adjacent a proximal end of the anchor opposite of the distal portion.

10. The anchor of claim 6, wherein the spring is configured to be selectively compressed and released to enable re-tensioning of the suture.
